(19) United States
(12) Patent Application Publication
(10) Pub. No.: US 2024/0112094 A1
(43) Pub. Date: Apr. 4, 2024

(54) Care Reservation and Booking System and Method

(71) Applicant: Purnima Sreenivasan, San Ramon, CA (US)

(72) Inventor: Purnima Sreenivasan, San Ramon, CA (US)

(21) Appl. No.: 18/463,247

(22) Filed: Sep. 7, 2023

Related U.S. Application Data
(60) Provisional application No. 63/404,419, filed on Sep. 7, 2022.

(51) Int. Cl. G06Q 10/02; G16H 40/20

(52) U.S. Cl. CPC G06Q 10/02; G16H 40/20

(57) ABSTRACT

A method of reservation and booking comprising: receiving information regarding a facility, receiving information regarding available quarters within said facility, receiving information regarding services available within said facility, storing said information regarding said facility, information regarding available quarters within said facility and services available within said facility in an indexed and searchable database; and verifying at least one certification associated with said facility via an external, third-party database. wherein at least one of information regarding said facility, information regarding available quarters within said facility and services available within said facility are searchable, wherein at least one of information regarding said facility, information regarding available quarters within said facility and services available within said facility is instantaneously updatable and wherein quarters within said facility are reservable.

### CROSS-REFERENCE TO RELATED APPLICATIONS

The present application claims the benefit of priority to prior-filed and co-pending U.S. Provisional Patent Application Ser. No. 63/404,419 filed Sep. 7, 2022 by Purnima Sreenivasan, the complete contents of which is hereby incorporated herein by reference.

### BACKGROUND

### Technical Field

The present device relates to the field of reservation and books systems and more particularly such for special needs facilities.

### Background

Searching for senior, adult and/or special needs care is currently conducted in an antiquated manner either via direct phone calls made to individual facilities and/or via brokers that work only with a restricted subset of facilities. Even when working through brokers, such are still required to call individual facilities to confirm availability and whether a facility has required capabilities or offers necessary services. Such is time consuming and frustrating. What is needed is a system and method for reservation handling and booking for seniors, adults and/or special needs care.

### BRIEF DESCRIPTION OF THE DRAWINGS

Further details of the present device are explained with the help of the attached drawings in which:

FIG. 1 depicts an overview of a system for reservation and booking.

FIG. 2 depicts a method of facility registration

FIG. 3 depicts a method of facility availability

FIG. 4 depicts a method of document verification

FIG. 5 depicts a method of room/care availability

FIG. 6 depicts an example of a care facility add page

FIG. 7 depicts an example of care facility staffing add page

FIG. 8 depicts an example of a facility/room information

FIG. 9 depicts an example of a facility capabilities page.

FIG. 10 depicts an example of an additional facility capabilities page.

FIG. 11 depicts an example of a room capabilities page.

FIG. 12 depicts an example of a client search page.

FIG. 13 depicts a networked computer system adapted and configured to perform the method(s) described herein.

FIG. 14 depicts an overview diagram of a system for care reservation and booking.

FIG. 15 depicts a method of registration and verification for a provider.

FIG. 16 depicts a method of registration and matching for a patient.

FIG. 17 depicts an alternate method of registration and matching of an insured patient.

FIG. 18 depicts a method of automatic contract generation and administration.

FIG. 19 depicts a method of automatic contract generation and administration for an insured patient.

FIG. 20 depicts a method of automatically predicting future care needs and presenting facilities meeting such anticipated care needs.

### DETAILED DESCRIPTION

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

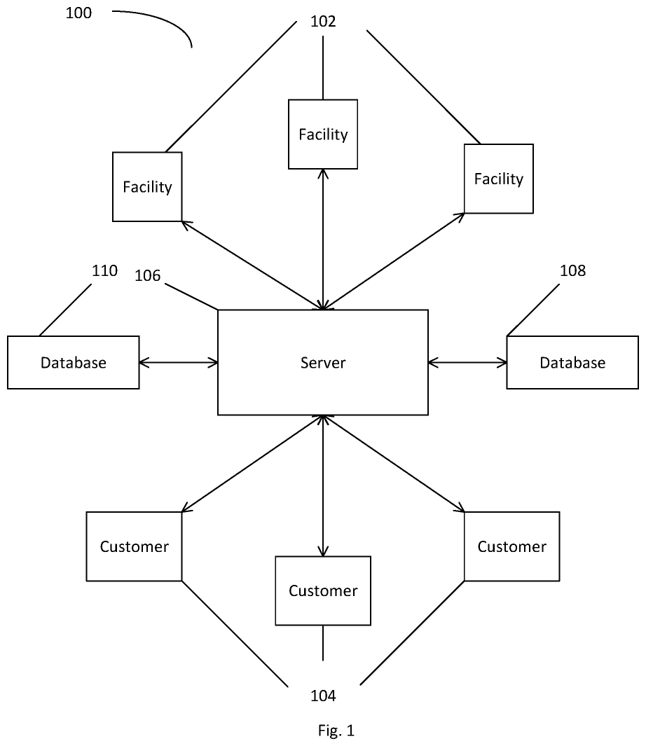

FIG. 1 depicts an overview of a system 100 for reservation and booking. In the embodiment depicted in FIG. 1, the system 100 is comprised of a plurality of facilities 102 and a plurality of customers 104 connected via a server 106 wherein the server 106 can be coupled with a database 108 to store information regarding the facilities and/or the customers and can be coupled with one or more databases 110 which contain information regarding facility and/or facility-staff credentials. In operation, a facility 100 can provide information regarding the facility, licensing, credential, capabilities and real-time room availability to a server 106 which can be stored within a database 108. Customers can register and search the database of real-time availability based on one or more criteria associates with the facilities and engage the facility to provide care for an individual based on their needs.

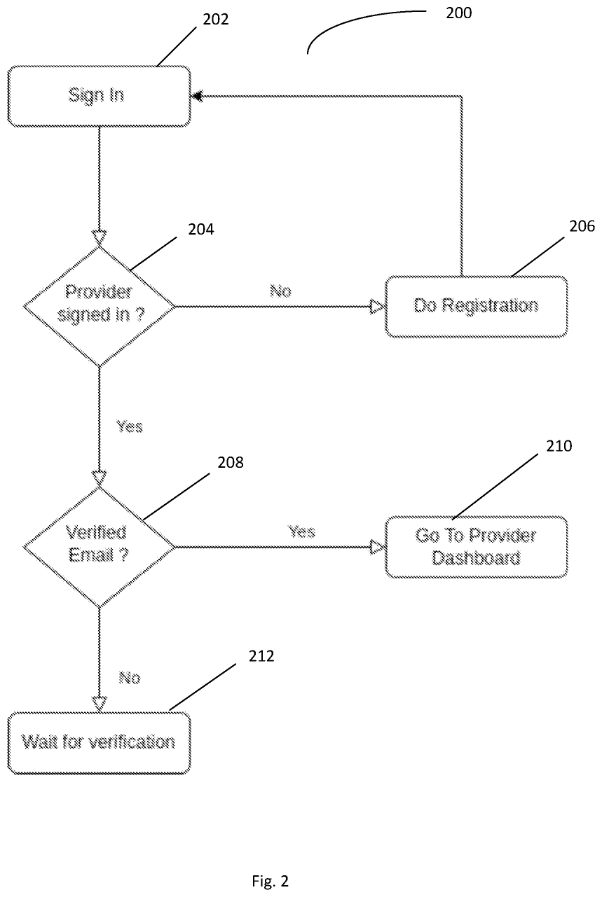

FIG. 2 depicts a method of facility registration 200. In step 202 a facility can sign into the system 100. In step 204 the system 100 can determine whether the facility is registered. If the system 100 determines that the facility is not registered, then the system can proceed to step 206 and a facility can register. If the system 100 determines that the facility can proceed to verification in step 208. If in step 208, it is determined that the facility's e-mail is registered, the system 100 can proceed to the provider dashboard in step 210. If it is determined that the facility's e-mail is not yet verified, the system 100 can proceed to step 212 wherein a facility's e-mail is verified.

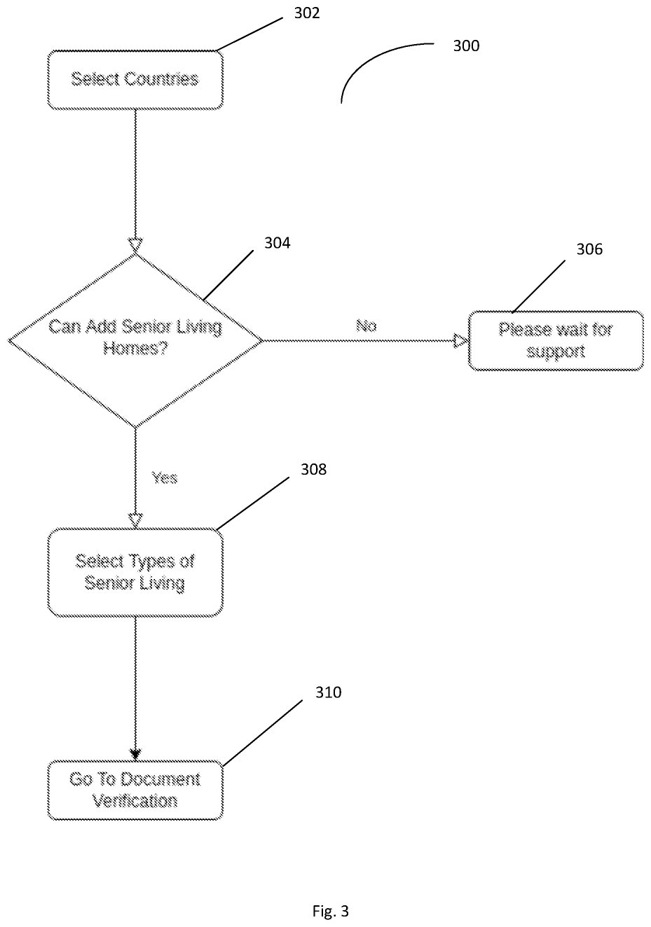

FIG. 3 depicts a method of facility availability 300. In step 302, the facility can select countries and then in step 304 the system 300 determines whether a facility is authorized to add senior living homes. If in step 304 it is determined that a facility is not authorized to add senior living homes, then the system 300 can proceed to step 306 and support can be provided. If in step 304 it is determined that a facility is authorized to add senior living homes, then the system 300 can proceed to step 308 and the type of senior living home can be entered and then in step 310 the system 300 can actively verify the documents provided by the facility.

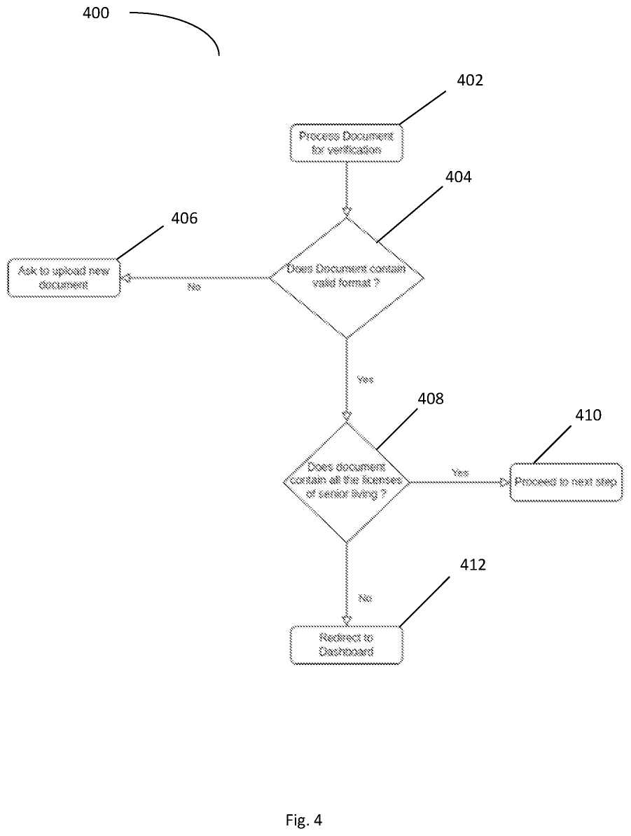

FIG. 4 depicts a method of document verification 400. In step 402 the documents provided by a facility can be received and processes to commence verification. If in step 404 the document is determined to have formatting that does not meet desired or required criteria, then the system can proceed to step 406 and a new document upload can be requested from the facility. If in step 404 it is determined that the document(s) meet required and/or desired formatting criteria, then the system can proceed to step 408 and document can be analyzed to determine whether the facility has required license(s). If in step 408 it is determined that the document does not indicate that the facility has required license(s), then the system 400 can indicate such and redirect a user to enter a new facility and/or document in step 412. If in step 408 it is determined that the document(s) indicate that the facility has requisite license(s), the facility can proceed to enter further data regarding the facility and availability in step 410.

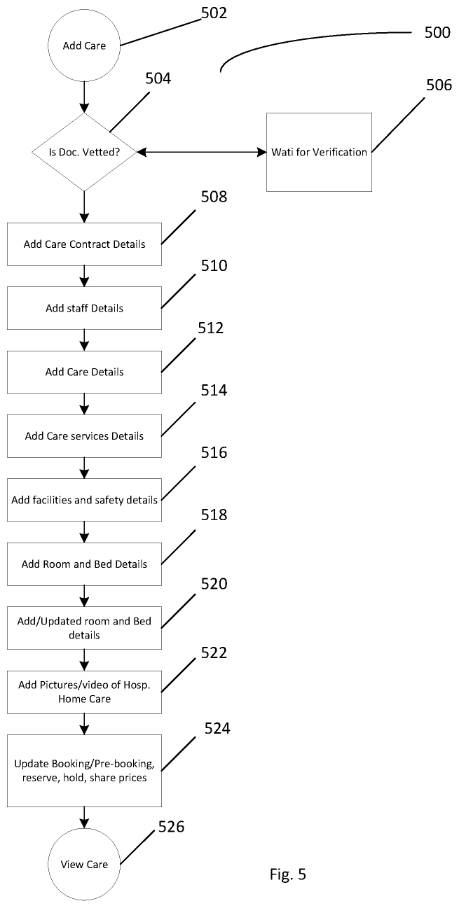

FIG. 5 depicts a method of room/care availability 500. In step 502, a facility can add information regarding room/care availability. In step 504 a determination is made regarding whether the document(s) are verified. If in step 504 it is determined that the document(s) is/are not verified, then a user can receive a message indicating that verification is pending or that verification is required prior to proceeding in step 506. If in step 504 it is determined that the document(s) is/are verified, then the system can proceed to step 508 an care contact details can be entered. Then in step 510 care staff details can be entered and/or updated and then in step 512 details regarding the care details can be entered and/or updated. (step 512) and care services can be entered and/or updated in step 514. In step 516 information regarding the care and facility safety details can be entered and/or updated. Then in steps 518 and 520, care and facility room availability can be entered and/or updated. In step 522, photographs and/or video of the facility, care and/or room can be entered and/or updated. Then in step 524, information regarding availability and pricing can be entered and/or updated. Finally, in step 526 the information entered/updated can be presented for verification by the user. All such information can then be stored in an indexed database for search by a user/consumer/client/patient. A user/consumer/client/patient can then search the database by providing customizable search criteria to locate facilities and/or care meeting their desired criteria.

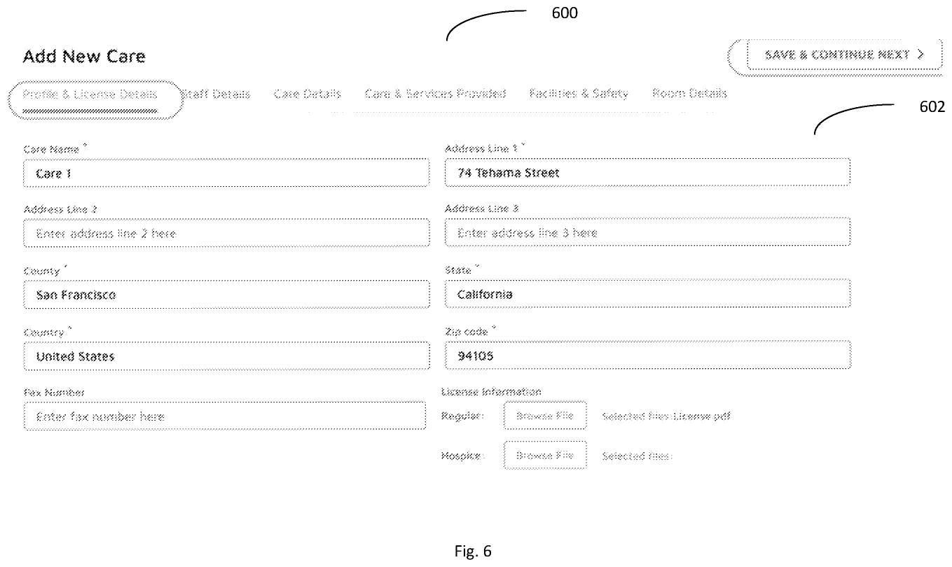

FIG. 6 depicts an example of a care facility add page 600 wherein essential contact and licensing information regarding the facility can be added and/or shown 602. Such information can comprise address, telephone number and facility licensing information and/or any other known, convenient and/or desired information.

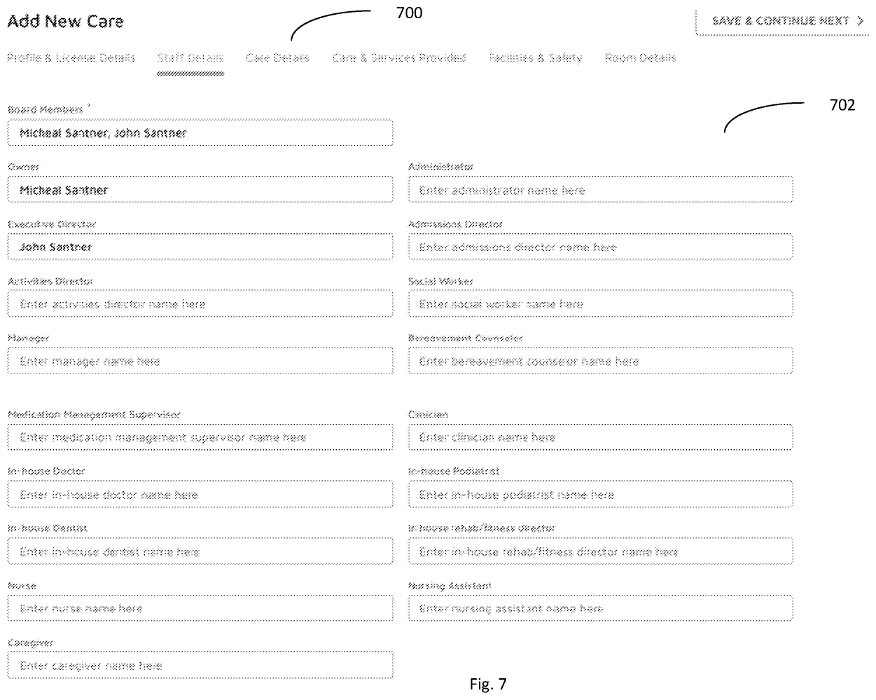

FIG. 7 depicts an example of care facility staffing add page 700 wherein information 702 regarding care can be entered and/or updated. Such information can comprise staffing information and/or any other known, convenient and/or desired information.

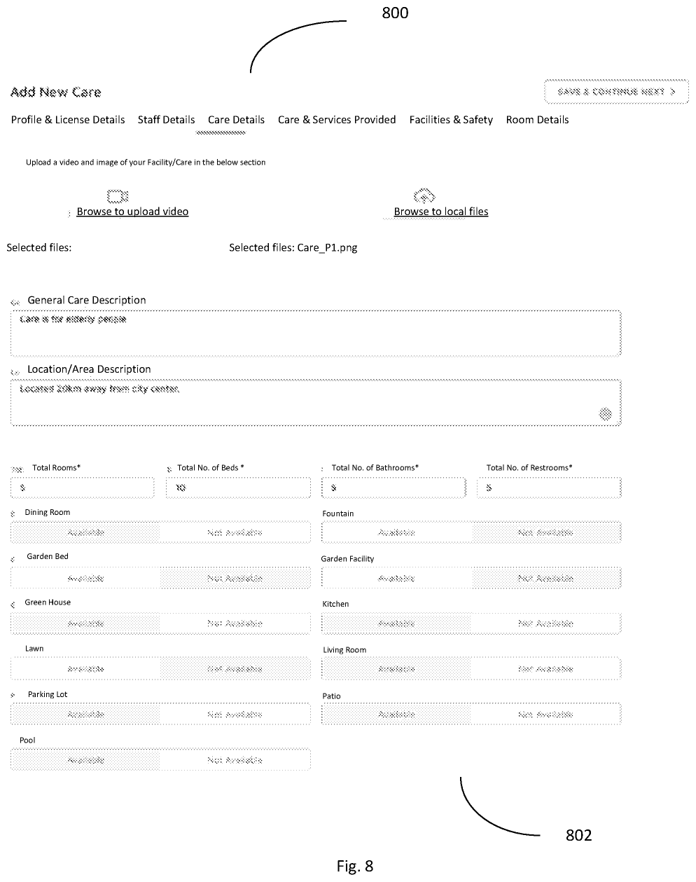

FIG. 8 depicts an example of a facility/room information 800 wherein information 802 regarding can comprise information such as, by way of non-limiting example, number of rooms, number of beds, number of bathrooms, number of restrooms, whether a facility has a dining room, garden, greenhouse, lawn, parking lot, pool, fountain, garden facility, kitchen, kitchen, living room, patio and/or any other known, convenient and/or desired feature. In some embodiments, the information can further comprise a customizable written description.

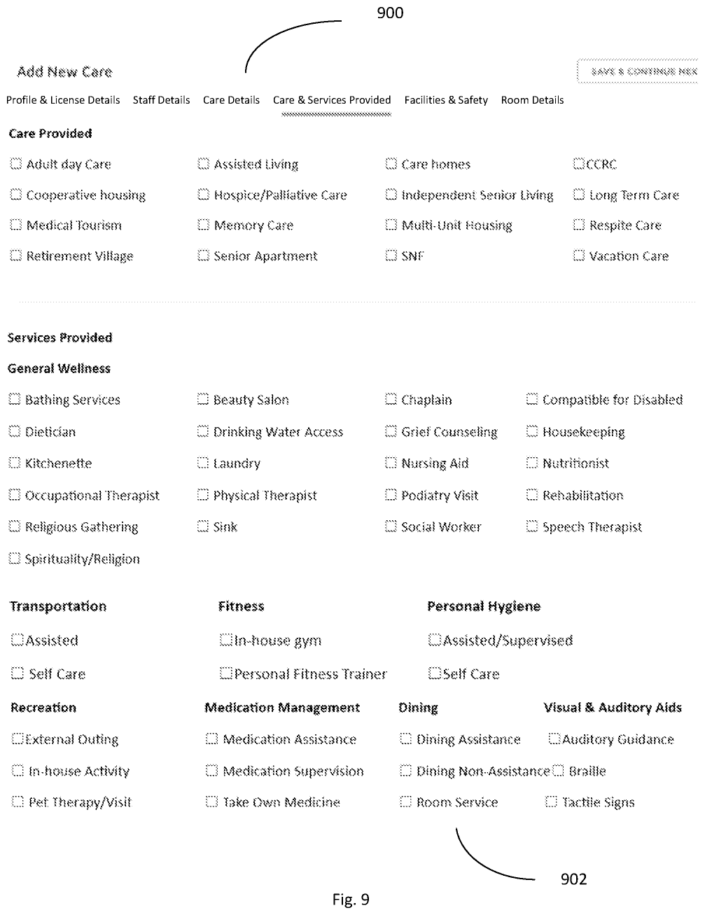

FIG. 9 depicts an example of a facility capabilities page 900 wherein information 902 regarding care and services available can be entered by a user. By way of non-limiting example, in some embodiments the care and/or services can comprise information regarding availability of adult day care, cooperative housing, medical tourism, retirement village, assisted living, hospice/palliative care, memory care, senior apartment, care homes, independent senior living, multi-unit housing, SNF, SNP, CCRC, long term care, respite care, vacation care, bathing services, dietician, kitchenette, occupational therapist, religious gathering, religion/spirituality, beauty salon, drinking water access, laundry, physical therapist, sink, Chaplin, grief counseling, nursing aid, podiatry visit, social working, compatible for disabled, housekeeping, nutritionist, rehabilitation, speech therapist, transportation, fitness, personal hygiene, recreation, medication management, dining, visual, auditory aids, and/or any other known convenient and/or desired information.

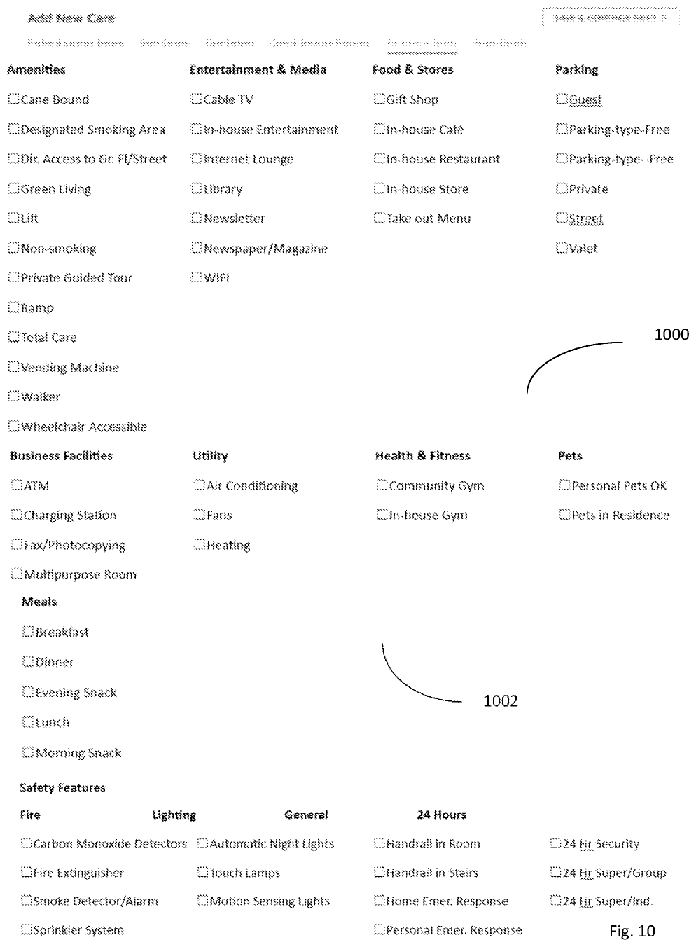

FIG. 10 depicts an example of an additional facility capabilities page 1000, wherein information 1002 regarding care and services available can be entered by a user. By way of non-limiting example, in some embodiments the care and/or services can comprise information regarding availability of amenities, entertainment/media, food/stores, parking, business facilities, utilities, health and fitness, pets, meals, safety information (fire, lighting, security) and/or any other known convenient and/or desired information.

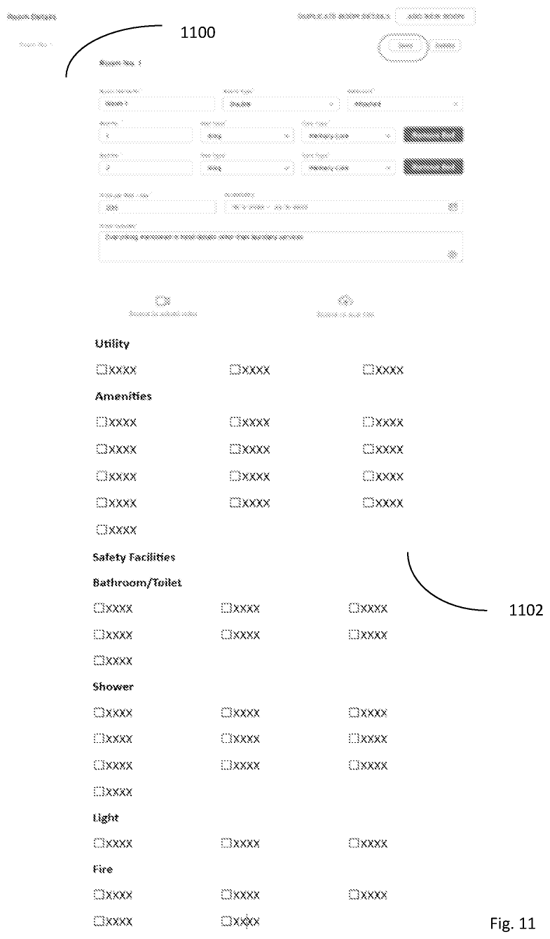

FIG. 11 depicts an example of a room capabilities page 1100 wherein information 1102 similar to that indicated in relation to FIGS. 8-10 can be provided, but in relation to a given room and information related to pricing and availability can be entered and/or updated.

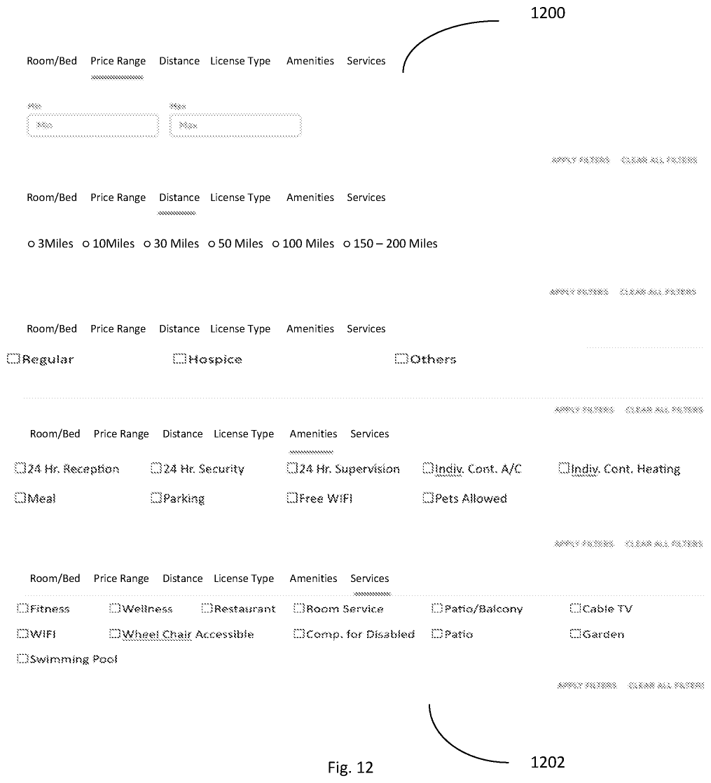

FIG. 12 depicts an example of a client search page 1200. By way of non-limiting example, in some embodiments the search criteria 1202 can comprise information regarding, geographic proximity, minimum/maximum pricing, licensure, amenities, room/bed type and availability, available services and/or any other known convenient and/or desired search criteria.

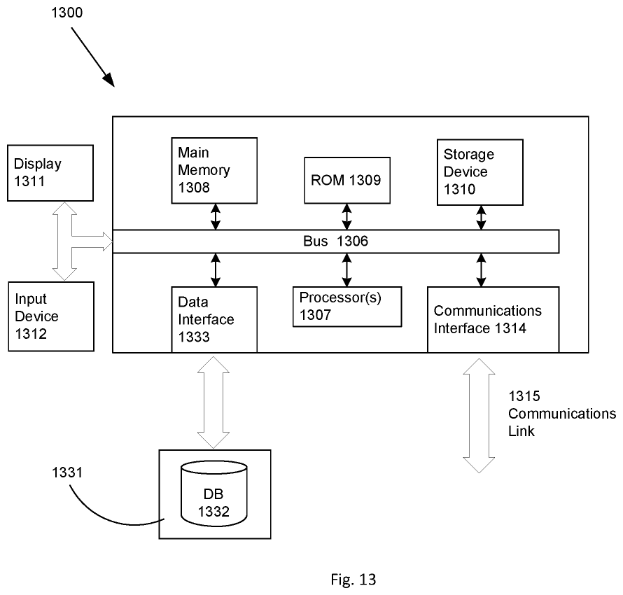

FIG. 13 depicts a networked computer system adapted and configured to perform the method(s) described herein.

The execution of the sequences of instructions required to practice the embodiments can be performed by a computer system 1300 as shown in FIG. 13. In an embodiment, execution of the sequences of instructions is performed by a single computer system 1300. According to other embodiments, two or more computer systems 1300 coupled by a communication link 1315 can perform the sequence of instructions in coordination with one another. Although a description of only one computer system 1300 will be presented below, however, it should be understood that any number of computer systems 1300 can be employed to practice the embodiments.

A computer system 1300 according to an embodiment will now be described with reference to FIG. 13, which is a block diagram of the functional components of a computer system 1300. As used herein, the term computer system 1300 is broadly used to describe any computing device that can store and independently run one or more programs.

Each computer system 1300 can include a communication interface 1314 coupled to the bus 1306. The communication interface 1314 provides two-way communication between computer systems 1300. The communication interface 1314 of a respective computer system 1300 transmits and receives electrical, electromagnetic or optical signals, that include data streams representing various types of signal information, e.g., instructions, messages and data. A communication link 1315 links one computer system 1300 with another computer system 1300. For example, the communication link 1315 can be a LAN, in which case the communication interface 1314 can be a LAN card, or the communication link 1315 can be a PSTN, in which case the communication interface 1314 can be an integrated services digital network (ISDN) card or a modem, or the communication link 1315 can be the Internet, in which case the communication interface 1314 can be a dial-up, cable or wireless modem.

A computer system 1300 can transmit and receive messages, data, and instructions, including program, i.e., application, code, through its respective communication link 1315 and communication interface 1314. Received program code can be executed by the respective processor(s) 1307 as it is received, and/or stored in the storage device 1310, or other associated non-volatile media, for later execution.

In an embodiment, the computer system 1300 operates in conjunction with a data storage system 1331, e.g., a data storage system 1331 that contains a database 1332 that is readily accessible by the computer system 1300. The computer system 1300 communicates with the data storage system 1331 through a data interface 1333. A data interface 1333, which is coupled to the bus 1306, transmits and receives electrical, electromagnetic or optical signals, that include data streams representing various types of signal information, e.g., instructions, messages and data. In embodiments, the functions of the data interface 1333 can be performed by the communication interface 1314.

Computer system 1300 includes a bus 1306 or other communication mechanism for communicating instructions, messages and data, collectively, information, and one or more processors 1307 coupled with the bus 1306 for processing information. Computer system 1300 also includes a main memory 1308, such as a random access memory (RAM) or other dynamic storage device, coupled to the bus 1306 for storing dynamic data and instructions to be executed by the processor(s) 1307. The main memory 1308 also can be used for storing temporary data, i.e., variables, or other intermediate information during execution of instructions by the processor(s) 1307.

The computer system 1300 can further include a read only memory (ROM) 1309 or other static storage device coupled to the bus 1306 for storing static data and instructions for the processor(s) 1307. A storage device 1310, such as a magnetic disk or optical disk, can also be provided and coupled to the bus 1306 for storing data and instructions for the processor(s) 1307.

A computer system 1300 can be coupled via the bus 1306 to a display device 1311, such as, but not limited to, a cathode ray tube (CRT) or a liquid-crystal display (LCD) monitor, for displaying information to a user. An input device 1312, e.g., alphanumeric and other keys, is coupled to the bus 1306 for communicating information and command selections to the processor(s) 1307.

According to one embodiment, an individual computer system 1300 performs specific operations by their respective processor(s) 1307 executing one or more sequences of one or more instructions contained in the main memory 1308. Such instructions can be read into the main memory 1308 from another computer-usable medium, such as the ROM 1309 or the storage device 1310. Execution of the sequences of instructions contained in the main memory 1308 causes the processor(s) 1307 to perform the processes described herein. In alternative embodiments, hard-wired circuitry can be used in place of or in combination with software instructions. Thus, embodiments are not limited to any specific combination of hardware circuitry and/or software.

The term “computer-usable medium,” as used herein, refers to any medium that provides information or is usable by the processor(s) 1307. Such a medium can take many forms, including, but not limited to, non-volatile, volatile and transmission media. Non-volatile media, i.e., media that can retain information in the absence of power, includes the ROM 1309, CD ROM, magnetic tape, and magnetic discs. Volatile media, i.e., media that can not retain information in the absence of power, includes the main memory 1308. Transmission media includes coaxial cables, copper wire and fiber optics, including the wires that comprise the bus 1306. Transmission media can also take the form of carrier waves; i.e., electromagnetic waves that can be modulated, as in frequency, amplitude or phase, to transmit information signals. Additionally, transmission media can take the form of acoustic or light waves, such as those generated during radio wave and infrared data communications.

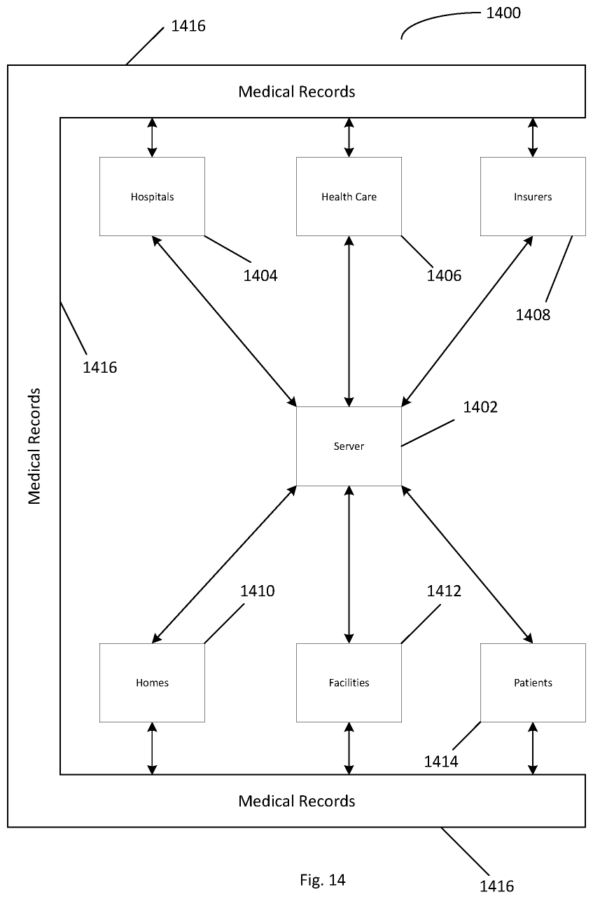

FIG. 14 depicts an overview diagram of a system 1400 for care reservation and booking. In the embodiment depicted in FIG. 14, system 1400 comprises at least one server 1402 in electronic communication with hospitals 1404, Health Care service providers 1406, insurers 1408, care homes 1410, facilities 1412 and patients 1404. Additionally, the hospitals 1404, Health Care service providers 1406, insurers 1408, care homes 1410, facilities 1412 and patients 1404 can be in electronic communication with various medical records databases/records 1416. In the embodiment depicted in FIG. 14, the server 1402 is not in direct communication with medical records databases/records 1416 as is agnostic to the contents of the medical records databases/records 1416. In some embodiments, server 1402 can be a plurality of servers in electronic communication.

In operation hospitals 1404, facilities 1412, housing 1410, care providers 1406 and other parties that serve elderly with specific care can sign up on the platform hosted on the server 1402 by adding their details, comprising, by way of non-limiting example, licenses, certifications, staff information, capabilities. These facilities' (1404, 1406, 1408, 1410, 1412. 1414) credentials for both the facilities and staff can then be automatically verified/confirmed by use of various online licensing/certification verification services such as Checkr®. If a facility fails screening/verification/confirmation of credentials/licensing, the facility can attempt re-registration after a prescribed period of time.

After a facility has cleared/passed the initial verification/screening process a facility can then add additional staffing and associated credentials and/or additional facilities and licensing credentials. Additional information can then be automatically verified through online licensing/certification verification services such as Checkr® prior to public viewing of such information.

Additionally, the facility and/or staff can provide various licenses/capabilities/staff associated with ICD-10 (International Classification of Disease 10) conditions, care types, cares provided in which these healthcare facilities specialize in and can be used in matching a patient elderly with their care needs through the online platform. While ICD-10 is the present standard/criteria employed, in alternate embodiments any known, convenient and/or desired care and/or classification systems can be employed.

These ICD-10 capabilities can then be verified per the care types and cares they provide and are certified for through certifications. By way of non-limiting example, if a hospital asserts they can provide wound care, then the hospital's assertion of wound care capabilities will be automatically verified though various online resources such as via Checkr®. Such verification process is applied to all asserted capabilities and all asserted licensures for both the facility, staff and equipment.

Care providers/facilities are pre-vetted and licensed and only certified care providers are permitted to sign up on the platform. In some embodiments, unvetted providers can sign up (only ones with 1-10 beds) but such will be appropriately flagged.

These vetted care providers/facilities with their specific ICD-10 codes they serve are connected and/or associated with each other stored via cloud storage, such that such information can be delivered to and/or access by the patient, elderly or families of elderly or discharge planners, social workers, case managers or anyone looking for specific cares or housing for their elderly when they sign in as a user and start searching for the right provider for the elderly anywhere globally.

All parties can communicate with each other through the platform internally via video communications, audio and share documents. In some embodiments, exchanged documents and/or exchanged records are shared via direct communication of the parties and/or are merely transmitted through the server with the server being agnostic to the content with no copies of such documents being stored on the server or on any database directly accessible via the server. Thus, while the server may be used for communication channels, records associated with the communications are not stored on or in any database associated with the server 1402. In this way, the server 1402 is HIPAA compliant and no medical records that are exchanged are stored anywhere on the server or technology associated with the server 1402.

Similarly, a patient and/or someone acting on behalf the patient can enter information regarding the patient's condition and care needs based on ICD-10 codes. One or more databases of various facilities can then be searched to match various criteria associated with client's location, budget, needs, and/or any other known, convenient and/or desired criteria to locate facilities that meet the patient's needs and other search criteria.

In some embodiments, the server 1402 can be used as standalone product or can integrate with care providers' internal technology via an appropriate Application Protocol Interface (API).

Upon matching and selection of a facility that meets a patient's needs/desires, a booking can be automatically created. In operation, a contract for care can be automatically generated based on the parties' needs and circulated for electronic signature. Additionally, in some embodiments the system 1400 can be enable booking, prebooking, bed holding, sharing, purchasing, selling and/or any other known, convenient and/or desired engagement means and/or mechanism.

The use of ICD-10 codes can facilitate future coding for insurance reimbursement, financial projections by third parties, save lives/research, data science tracking each condition across the world, and help streamline care costs and cares for each individual elderly at any given time in their aging needs wants haves.

Additionally, in some embodiments, the server 1402 can provide reminder regarding licensing/certification expiration and/or provide access to education for both providers and consumers to meet their needs for licensing and certifications in order to provide the highest care through third party integrations and product offerings as well.

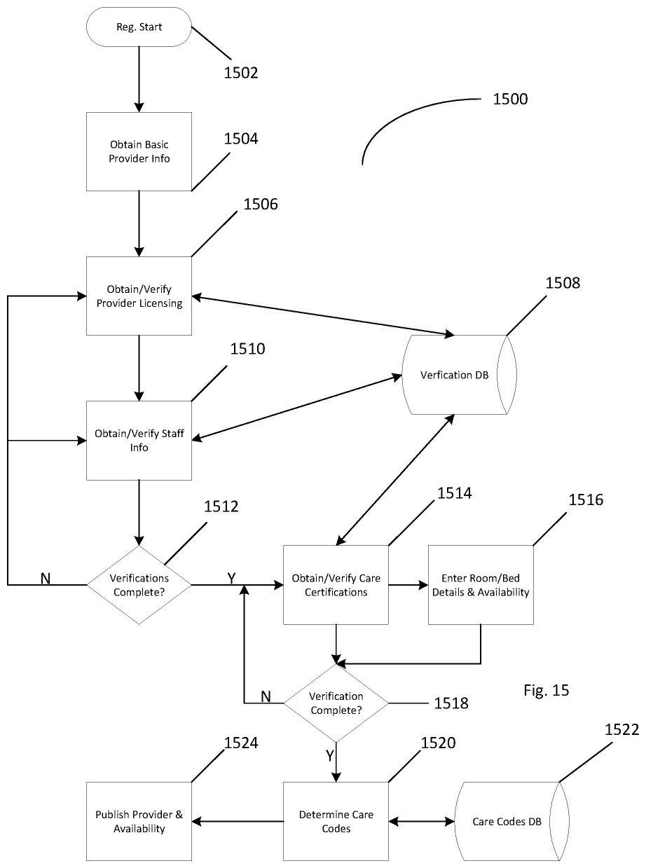

FIG. 15 depicts a method of registration and verification for a provider 1500. The method or registration is initiated at step 1502 and in step 1504, the provider enters basic information regarding the provider such as, by way of non-limiting example, username, password and essential contract information. In step 1506, a provider can enter information regarding the facility and capabilities of the facility which can then be automatically verified via electronic communication with one or more online verification databases 1508. In step 1510, licensing, education, and training information regarding staff members can then be entered and automatically verified via electronic communication with one or more online verification databases 1508.

In step 1512 verification of the provider and staff is confirmed. If one or more licenses/certifications/training/education verifications fails, the method can return to step 1506 and/or 1510 to enable verification/correction of information to be recheck against the verification databases 1508. Method 1500 proceeds to step 1514 after all staff and provider information has been verified. In some embodiments, the number of attempts at verification may be restricted and a facility and/or staff member may be prohibited from re-attempting verification for a period of time, if verification fails a certain number of times.

In step 1514, the capabilities of the facility and/or staff based on ICD-10 codes and/or any other known, convenient and/or desired classification system can be automatically verified via electronic communication with one or more online verification databases 1508.

In step 1516, after verification in step 1514, information regarding room and/or bed availability, details and pricing can be entered. Then in step 1518, if verification from step 1514 is complete, care codes (in some embodiments associated with IDC 10 codes) can be determined, in some instances by accessing an external database 1522. After care codes are associated with the facility and room/bed availability, a listing can be published and become searchable.

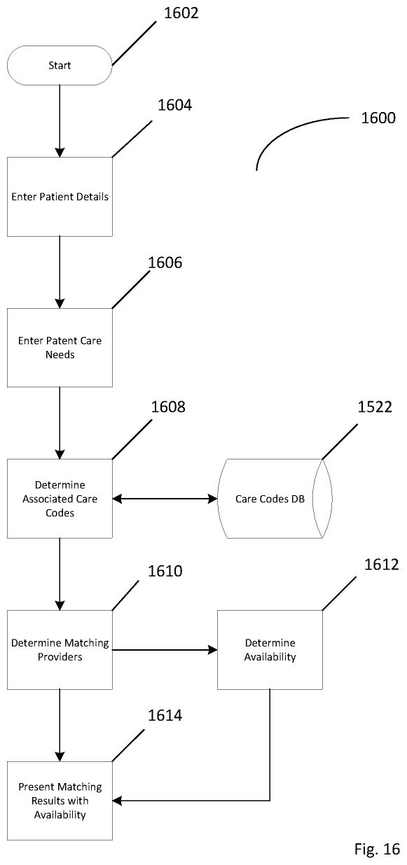

FIG. 16 depicts a method of registration and matching 1602 for a patient. In the embodiment depicted in FIG. 16, the method of patient registration and matching can be initiated in step 1602. Patient information can be entered in step 1604 comprising information, such as, a username, password, contact information then in step 1606 patient care needs can be entered and then care codes can be determined in step 1608 from the patient care needs identified in step 1606. Again, patient care codes can be determined either internally and/or by electronic communication a care codes database 1522.

In step 1610 providers can be identified that match care code criteria and patent needs and desires and in step 1612, availability of providers can be determined before results and availability are presented in step 1614.

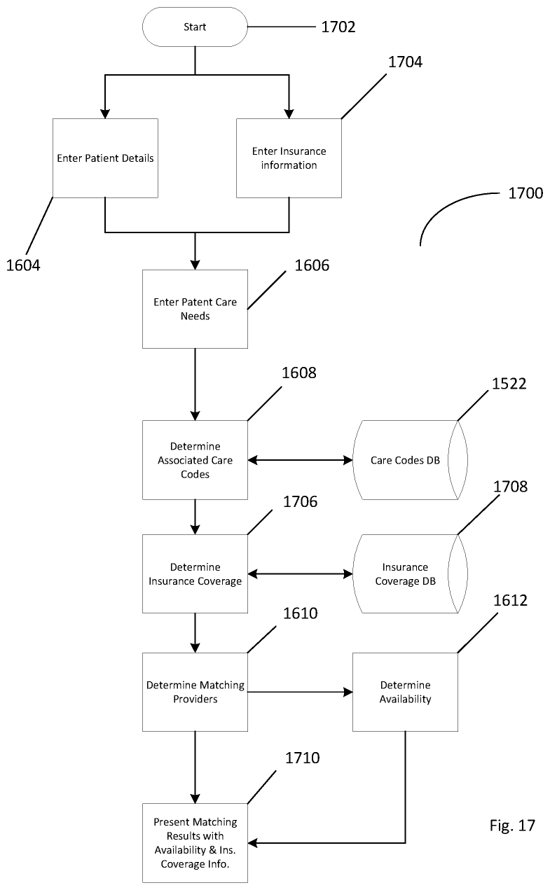

FIG. 17 depicts an alternate method of registration and matching of an insured patient 1700. In the embodiment depicted in FIG. 17, the method of patient registration and matching can be initiated in step 1702. Patient information can be entered in step 1604 comprising information, such as, a username, password, contact information. Additionally, in step 1704 patient insurance information can be entered which can comprise information regarding coverage/benefit information. Then in step 1606 patient care needs can be entered and care codes can be determined in step 1608 from the patient care needs identified in step 1606 and insurance coverage/benefits information obtained in step 1704. Again, patient care codes can be determined either internally and/or by electronic communication a care codes database 1522.

In step 1706, insurance coverage benefits can be determined and then in step 1610 providers can be identified that match care code criteria, patent needs and desires and insurance coverage/benefits. In some embodiments, coverage benefits can be determined by electronic communication with one or more insurance coverage/benefits databases 1708. Then in step 1612, availability of providers can be determined along with available benefits and results, availability and coverage/benefits are presented in step 1710.

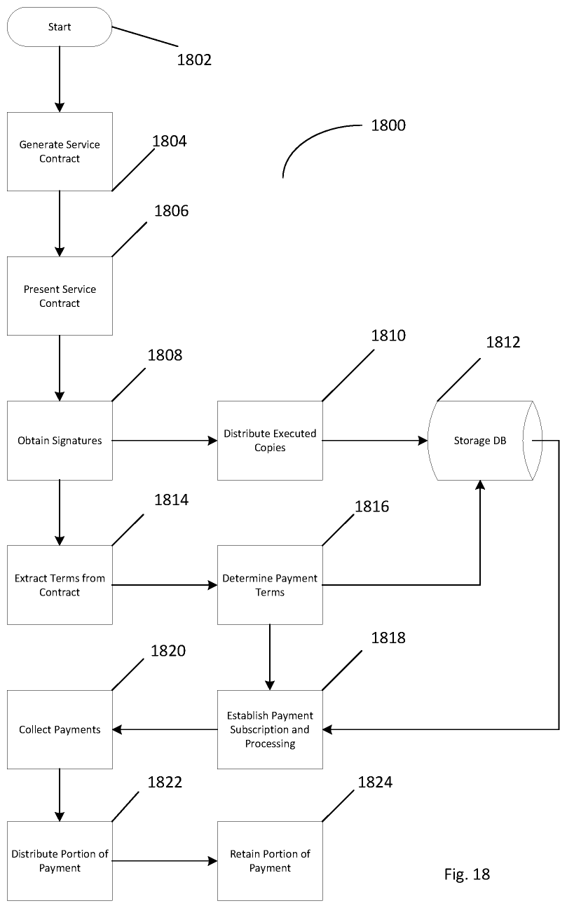

FIG. 18 depicts a method of automatic contract generation and administration 1800. In step 1802 the method of automatic contract generation and administration 1800 can be initiated. In step 1804 a services contract can be instantiated based at least in part on patient care needs and matching provider availability and the instantiated contract can be presented to the parties in step 1806. After signatures of the parties are obtained, electronically or otherwise, the executed contract can be distributed to the parties in step 1810 and stored in a database in step 1812. Terms of the contract can be extracted from the executed agreement in step 1814 and payment terms determined in step 1816. Information regarding payment terms can then be stored and/or updated in the storage database 1812. Depending on the terms of the contract, payment can then be initiated and/or an ongoing payment program can be established in step 1818 and payments can be collected for the services to be provided by the provider in step 1820. In some embodiments, a portion of the payment can be collected as a fee in step 1822 and the reminder of the payments collected can be distributed to the provider in step 1824. In some embodiments, step 1822 may be absent.

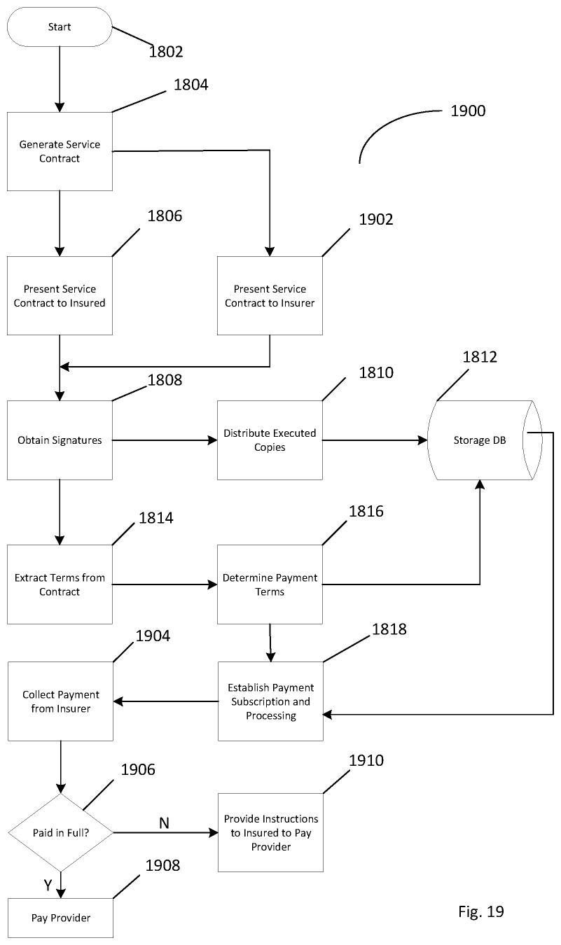

FIG. 19 depicts a method of automatic contract generation and administration for an insured patient 1900. In step 1802 the method of automatic contract generation and administration 1800 can be initiated. In step 1804 a services contract can be instantiated based at least in part on patient care needs and matching provider availability and the instantiated contract can be presented to the parties in step 1806 and presented to the insurer in step 1902. After signatures of the parties and the insurer are obtained, electronically or otherwise, the executed contract can be distributed to the parties and the insurer in step 1810 and stored in a database in step 1812. Terms of the contract can be extracted from the executed agreement in step 1814 and payment terms determined in step 1816. Information regarding payment terms can then be stored and/or updated in the storage database 1812. Depending on the terms of the contract, payment can then be initiated and/or an ongoing payment program can be established in step 1818 and payments can be collected from the insurer for the services to be provided by the provider in step 1904. In step 1906 it can be determined whether the insurance coverage/benefits cover all of the fees due to the service provider. If payments from the insurer cover all amounts due to provider, then the provider is paid in step 1908 and the method ends. If in step 1906 it is determined that benefits/coverage from the insurer do not cover all amounts due to the service provider, then in step 1910 a shortfall payment due notice is sent to the insured indicating the amount due by the insured and the provider is paid in step 1908.

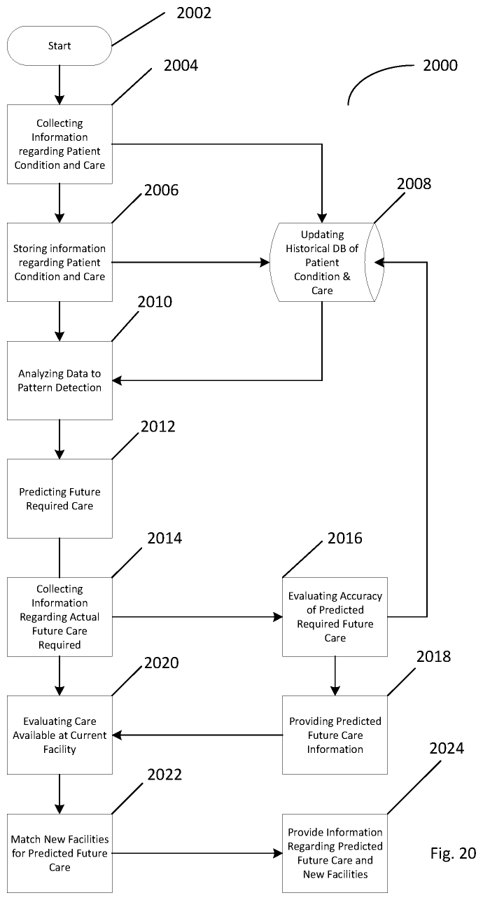

FIG. 20 depicts a method of automatically predicting future care needs and presenting facilities meeting such anticipated care needs 2000. In step 2002 the method is initiated. In step 2004, information regarding a patient's condition and care needs is collected and stored in a database 2008 in step 2006. In some embodiments care needs can be stored in association with ICD-10 codes. However, in alternate embodiments, other coding and/or categorization techniques and/or systems can be employed. In step 2010 data regarding associated codes can be analyzed to detect patterns and/or progression of patients which present with particular care/needs. Based on pattern/progression predictions, predicted future care needs can be identified for patients in step 2012. In step 2014 patients' actual needs over time can be assessed in step 2016 and used to evaluate the accuracy of the predicted future care from step 2012. That accuracy data can then be update and train the database 2008 such that more accurate predictions regarding anticipated future care can be made.

In step 2018 predicted future care can be identified based on a patient's current condition and care needs and then in step 2020 care provided by a current provider can be evaluated to determine if it will meet anticipated/predicted future care needs. Additional searching can then be automatically run based on a patient's current and anticipated future care needs and new/future/alternate providers can be identified based on the patient's anticipated/predicted future needs in step 2022. Then in step 2024 information regarding anticipated future needs and providers capable of providing such anticipated future care can be presented.

In the foregoing specification, the embodiments have been described with reference to specific elements thereof. It will, however, be evident that various modifications and changes can be made thereto without departing from the broader spirit and scope of the embodiments. For example, the reader is to understand that the specific ordering and combination of process actions shown in the process flow diagrams described herein is merely illustrative, and that using different or additional process actions, or a different combination or ordering of process actions can be used to enact the embodiments. The specification and drawings are, accordingly, to be regarded in an illustrative rather than restrictive sense.

It should also be noted that the present invention can be implemented in a variety of computer systems. The various techniques described herein can be implemented in hardware or software, or a combination of both. Preferably, the techniques are implemented in computer programs executing on programmable computers that each include a processor, a storage medium readable by the processor (including volatile and non-volatile memory and/or storage elements), at least one input device, and at least one output device. Program code is applied to data entered using the input device to perform the functions described above and to generate output information. The output information is applied to one or more output devices. Each program is preferably implemented in a high-level procedural or object oriented programming language to communicate with a computer system. However, the programs can be implemented in assembly or machine language, if desired. In any case, the language can be a compiled or interpreted language. Each such computer program is preferably stored on a storage medium or device (e.g., ROM or magnetic disk) that is readable by a general or special purpose programmable computer for configuring and operating the computer when the storage medium or device is read by the computer to perform the procedures described above. The system can also be considered to be implemented as a computer-readable storage medium, configured with a computer program, where the storage medium so configured causes a computer to operate in a specific and predefined manner. Further, the storage elements of the exemplary computing applications can be relational or sequential (flat file) type computing databases that are capable of storing data in various combinations and configurations.

Although exemplary embodiments of the invention have been described in detail and in language specific to structural features and/or methodological acts above, it is to be understood that those skilled in the art will readily appreciate that many additional modifications are possible in the exemplary embodiments without materially departing from the novel teachings and advantages of the invention. Moreover, it is to be understood that the subject matter defined in the appended claims is not necessarily limited to the specific features or acts described above. Accordingly, these and all such modifications are intended to be included within the scope of this invention construed in breadth and scope in accordance with the appended claims.

What is claimed:

1. A reservation and booking system comprising:
first memory adapted and configured to receive information regarding a facility;
second memory adapted and configured to receive information regarding available quarters within said facility;
third memory adapted and configured to receive information regarding services available within said facility;
storage adapted and configured to store said information regarding said facility, information regarding available quarters within said facility and services available within said facility in an indexed and searchable database; and
network interface components adapted and configured such that at least one certification associated with said facility can be verified via an external, third-party database;
wherein at least one of information regarding said facility, information regarding available quarters within said facility and services available within said facility are searchable;
wherein at least one of information regarding said facility, information regarding available quarters within said facility and services available within said facility is instantaneously updatable; and
wherein quarters within said facility are reservable.

2. The reservation and booking system of claim 1 wherein said facility is associated with care codes associated with certifications and abilities of said facility to provide specific care.

3. The reservation and booking system of claim 2 wherein said searchable database can be searched based at least on said care codes.

4. The reservation and booking system of claim 3 wherein said care codes are ICD-10 codes.

5. The reservation and booking system of claim 1 wherein said quarters is associated with care codes associated with certifications and abilities associated with said quarters to provide specific care.

6. The reservation and booking system of claim 2 wherein said searchable database can be searched based at least on said care codes.

7. The reservation and booking system of claim 3 wherein said care codes are ICD-10 codes.

8. A method of reservation and booking comprising:
receiving information regarding a facility;
receiving information regarding available quarters within said facility;
receiving information regarding services available within said facility;
storing said information regarding said facility, information regarding available quarters within said facility and services available within said facility in an indexed and searchable database; and
verifying at least one certification associated with said facility via an external, third-party database;
wherein at least one of information regarding said facility, information regarding available quarters within said facility and services available within said facility are searchable;
wherein at least one of information regarding said facility, information regarding available quarters within said facility and services available within said facility is instantaneously updatable; and
wherein quarters within said facility are reservable.

9. The method of reservation and booking of claim 8 wherein said facility is associated with care codes associated with certifications and abilities of said facility to provide specific care.

10. The method of reservation and booking of claim 9 wherein said searchable database can be searched based at least on said care codes.

11. The method of reservation and booking of claim 10 wherein said care codes are ICD-10 codes.

12. The method of reservation and booking of claim 8 wherein said quarters is associated with care codes associated with certifications and abilities associated with said quarters to provide specific care.

13. The method of reservation and booking of claim 12 wherein said searchable database can be searched based at least on said care codes.

14. The method of reservation and booking of claim 13 wherein said care codes are ICD-10 codes.
